(19) United States
(12) Patent Application Publication
(10) Pub. No.: US 2015/0150544 A1
(43) Pub. Date: Jun. 4, 2015

(54) Apparatuses and Methods for Endoscope and/or With Tool Actuated by Shape Memory Alloy Element

(71) Applicants: Dobromir Filip, Calgary (CA); Martin Mintchev, Calgary (CA); Christopher Andrews, Calgary (CA); Orly Yadid-Pecht, Calgary (CA)

(72) Inventors: Dobromir Filip, Calgary (CA); Martin Mintchev, Calgary (CA); Christopher Andrews, Calgary (CA); Orly Yadid-Pecht, Calgary (CA)

(73) Assignee: UTI LIMITED PARTNERSHIP, Calgary, AB (CA)

(21) Appl. No.: 14/129,853

(22) PCT Filed: Jun. 29, 2012
(86) PCT No.: PCT/IB2012/001716

§ 371 Date: Dec. 9, 2014

Related U.S. Application Data
(60) Provisional application No. 61/502,682, filed on Jun. 29, 2011.

(51) Int. Cl. A61B 10/04; A61B 5/15; A61M 5/158

(52) U.S. Cl. CPC A61B 10/04; A61M 5/158; A61B 5/150412; A61B 2010/0208

(57) ABSTRACT

Apparatuses and methods for capsule endoscopes with tools actuated by a combination of a shape memory alloy (SMA) element and a biasing element (e.g., spring). Apparatuses and methods for biopsies actuated by an SMA element.

### CROSS-REFERENCE TO RELATED APPLICATION

This application claims priority to U.S. Provisional Patent Application No. 61/502,682, filed Jun. 29, 2011, which is incorporated by reference in its entirety.

### BACKGROUND

1. Field of the Invention

The present invention relates generally to actuation of small-scale tools with shape-memory alloy. More particularly, but not by way of limitation, the present invention relates to tools and SMA actuation mechanisms for tools in capsule endoscopes.

2. Background Information

Endoscopic capsules may be used to investigate the digestive (gastrointestinal) tract of an animal (e.g., a human). One example of such a capsule is disclosed in US 2006/0152309. Such capsules may be passively controlled or steered. For example, such capsules may comprise magnetic material and may be controlled or steered by the presence of magnetic fields generated or controlled by a user (e.g., outside the body of an animal in which a capsule is disposed). Such capsules may also include a source of locomotion or steering within or on the capsule (e.g., a source of active locomotion).

### SUMMARY

The present disclosure includes embodiments of apparatuses, tools for capsule endoscopes, and methods.

Some embodiments of the present apparatuses comprise: a capsule configured to be disposed in the digestive tract of an animal; a shape memory alloy (SMA) element configured to change at least one of its dimensions if a current is applied to the SMA element, the SMA element coupled to the capsule; a biasing element (e.g., a spring or any other suitable elastic element) coupled to the capsule; and a tool coupled to the SMA element and the biasing element such that a current can be applied to the SMA element to shift the tool from a first configuration to a second configuration, and if the tool is in the second configuration the current can be removed to shift the tool from the second configuration to the first configuration.

In some embodiments, the biasing element comprises a spring. In some embodiments, the biasing element comprises rubber or polymer. In some embodiments, a longitudinal axis of the biasing element is parallel to a longitudinal axis of the SMA element. In some embodiments, the biasing element is coaxial with the SMA element.

In some embodiments, the SMA element is configured to expand if a current is applied to the SMA element. In some embodiments, the capsule further comprises: a power source configured to be coupled to the SMA element to apply a current to the SMA element. In some embodiments, the tool is retracted in the first configuration, and the tool is extended in the second configuration. In some embodiments, the apparatus is configured such that if the tool is in the second configuration, the biasing element applies a force biasing the tool toward the first configuration. In some embodiments, the tool comprises a base and a piston, and the piston is extended relative to the base in the second configuration. In some embodiments, the SMA element is configured such that application of the current to the SMA element will cause the at least one dimension of the SMA element to increase and apply a force to the piston toward the second configuration, and if the piston is in the second configuration the biasing element biases the piston toward the first configuration.

Some embodiments further comprise: a cord coupled to and between the piston and the biasing element. In some embodiments, a longitudinal axis of the biasing element is not co-linear with a longitudinal axis of the piston. In some embodiments, the base of the tool comprises an opening aligned with a longitudinal axis of the piston, and the cord passes through the opening and is coupled to the center of a distal end of the piston.

In some embodiments, the tool is configured to contact tissue to deliver a therapeutic agent or retrieve a tissue sample. In some embodiments, the tool comprises a needle coupled to a distal end of the piston. In some embodiments, the base of the tool is coupled to the piston of the tool such that: relative longitudinal motion between the piston and base is permitted; and relative lateral motion between the piston and base is substantially prevented. In some embodiments, at least a portion of the SMA element is disposed within the piston. In some embodiments, the tool comprises a biopsy needle coupled to a distal end of the piston. In some embodiments, the apparatus is configured such that the piston can be actuated to apply a force of at least 20 grams force (gf). In some embodiments, the apparatus is configured such that the tool can be actuated to apply a force of at least 20 grams force (gf). In some embodiments, a longitudinal axis of the biasing element is parallel to a longitudinal axis of the SMA element. In some embodiments, the biasing element is coaxial with the SMA element. In some embodiments, the capsule has a length of less than 40 mm and a diameter of less than 15 mm. In some embodiments, the capsule has a length of less than 32 mm (e.g., 15-20 mm) and a transverse dimension of less than 12 mm (e.g., 6-10 mm).

Some embodiments of the present methods comprise: applying an electric current to the SMA element of an embodiment of the present apparatuses that is disposed in the digestive tract of an animal. In some embodiments, the apparatus comprises a tool with a biopsy needle, and the method further comprises: actuating the tool to insert the biopsy needle into target tissue of the animal. In some embodiments, the method further comprises: retrieving a tissue sample from the biopsy needle.

Some embodiments of the present biopsy apparatuses comprises: a body; a shape memory alloy (SMA) element coupled to the body and configured to change at least one of its dimensions if a current is applied to the SMA element, the SMA element coupled to the capsule; and a tool configured to contact tissue to retrieve a tissue sample, the tool coupled to the SMA element such that a current can be applied to the SMA element to shift the tool from a first configuration to a second configuration, and if the tool is in the second configuration the current can be removed to shift the tool from the second configuration to the first configuration. In some embodiments, the SMA element is configured to expand if a current is applied to the SMA element. Some embodiments further comprise: a power source configured to be coupled to the SMA element to apply a current to the SMA element. In some embodiments, the tool is retracted in the first configuration, and the tool is extended in the second configuration. In some embodiments, the tool comprises a base, and a piston, where the piston is extended relative to the base in the second configuration. In some embodiments, the SMA element is configured such that application of the current to the SMA element will cause the at least one dimension of the SMA element to increase and apply a force to the piston toward the second configuration. In some embodiments, at least a portion of the SMA element is disposed within the piston. In some embodiments, the tool comprises a biopsy needle coupled to a distal end of the piston. In some embodiments, the apparatus is configured such that the piston can be actuated to apply a force of at least 20 grams force (gf). Some embodiments further comprise: a biasing element coupled to the tool and to the body and configured to apply a force urging the tool toward the first configuration. In some embodiments, a longitudinal axis of the biasing element is parallel to a longitudinal axis of the SMA element. In some embodiments, the biasing element is coaxial with the SMA element.

Some embodiments of the present methods comprise: applying an electric current to the SMA element of an embodiment of the present apparatuses that is disposed within an animal such that the tool obtains a tissue or fluid sample from the animal. In some embodiments, the tissue or fluid sample is obtained from the digestive tract of the animal.

In any embodiment of the present disclosure, the term “substantially” may be substituted with “within [a percentage] of” what is specified, where the percentage includes 5, 10, and/or 15 percent.

Any embodiment of any of the present systems and/or methods can consist of or consist essentially of—rather than comprise/include/contain/have—any of the described steps, elements, and/or features. Thus, in any of the claims, the term “consisting of” or “consisting essentially of” can be substituted for any of the open-ended linking verbs recited above, in order to change the scope of a given claim from what it would otherwise be using the open-ended linking verb.

Details associated with the embodiments described above and others are presented below.

### BRIEF DESCRIPTION OF THE DRAWINGS

The following drawings illustrate by way of example and not limitation. For the sake of brevity and clarity, every feature of a given structure is not always labeled in every figure in which that structure appears. Identical reference numbers do not necessarily indicate an identical structure. Rather, the same reference number may be used to indicate a similar feature or a feature with similar functionality, as may non-identical reference numbers.

FIG. 1 depicts a side view of one of the present apparatuses, a capsule endoscope having a tool.

FIG. 2 depicts a perspective cross-sectional view of a tool for use in apparatuses such as the capsule of FIG. 1, with the tool shown in a compressed configuration.

FIG. 3 depicts a perspective cross-sectional view of the tool of FIG. 2, with the tool shown in an expanded configuration.

FIG. 4 depicts an end cross-sectional view of the tool of FIG. 2, taken along the line 4-4 of FIG. 5.

FIG. 5 depicts a side cross-sectional view of the tool in the compressed configuration of FIG. 2.

FIG. 6 depicts a side cross-sectional view of the tool in the expanded configuration of FIG. 3.

FIG. 7 depicts a perspective view of a biopsy module including an embodiment of the present tools for use in apparatuses such as the capsule of FIG. 1.

FIG. 8 depicts a perspective view of a second embodiment of the present capsule endoscopes that includes the biopsy module of FIG. 7.

FIG. 9 depicts a side cross-sectional view of a third embodiment of the present tools.

### DESCRIPTION OF ILLUSTRATIVE EMBODIMENTS

The term “coupled” is defined as connected, although not necessarily directly, and not necessarily mechanically; two items that are “coupled” may be integral with each other. The terms “a” and “an” are defined as one or more unless this disclosure explicitly requires otherwise. The terms “substantially,” “approximately,” and “about” are defined as largely but not necessarily wholly what is specified, as understood by a person of ordinary skill in the art.

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a method that “comprises,” “has,” “includes” or “contains” one or more steps possesses those one or more steps, but is not limited to possessing only those one or more steps. Likewise, an apparatus that “comprises,” “has,” “includes” or “contains” one or more elements possesses those one or more elements, but is not limited to possessing only those elements. For example, in an apparatus that comprises a capsule, a shape memory alloy (SMA) element, a biasing element, and a tool, the apparatus includes the specified elements but is not limited to having only those elements. For example, such an apparatus could also include a cord coupled to the tool and the biasing element.

Further, a device or structure that is configured in a certain way is configured in at least that way, but it can also be configured in other ways than those specifically described.

The present disclosure includes apparatuses and methods for tissue and fluid sampling, such as can be implemented or included in a capsule endoscope (CE). Locomotion of such a capsule endoscope may be accomplished actively or passively. In some embodiments, a remotely activated shape memory alloy (SMA) element is configured to actuate or extend a biopsy needle (e.g., a micro-needle) such that a tissue sample can be obtained from any point within one 360° circumference of the capsule, such as, for example, a sample from nearly any point on an internal organ of an animal in which the capsule is disposed. For example, once a biopsy site is identified, the CE may be moved into position with the (longitudinal axis of the) micro-needle perpendicular to the surface of the sample area. Different approaches can be utilized to reach this biopsy site, such as magnetic steering or an active locomotion. As such, embodiments of the present tools can be incorporated into capsule endoscopes that are configured to be magnetically steered (e.g., can comprise one or more magnetic and/or magnetically reactive materials of sufficient mass to permit magnetic steering of the capsule). At the beginning of the biopsy, the micro-needle is aligned with the outer edge of the CE. Once the SMA element is activated to perform the biopsy, the micro-needle punctures the targeted tissue, and then is automatically retracted into the assembly. ensnaring the sample in a cavity of the needle, in accordance with the various features described in additional detail below. Additionally, in the present embodiments, the stroke or distance to which a tool (e.g., biopsy needle) is deployed can be adjusted such that a user can limit the deployment such that the tool punctures tissue, punctures only an outer layer of tissue, or does not puncture tissue (e.g., to obtain a fluid sample). By way of further examples, the tools of the present embodiments can be used and/or configured to deploy (e.g., in place of or in addition to a sampling needle): a sensing device (which may benefit from the ability to be deployed nearer to tissue), needles and micro-spikes (e.g., needle structure with varying cavities that may entrap sampled tissue), a pH-sensitive layer for assessing tissue pH (e.g., as may be important for cancers), an imaging unit (e.g., infrared, fluorescent sensors).

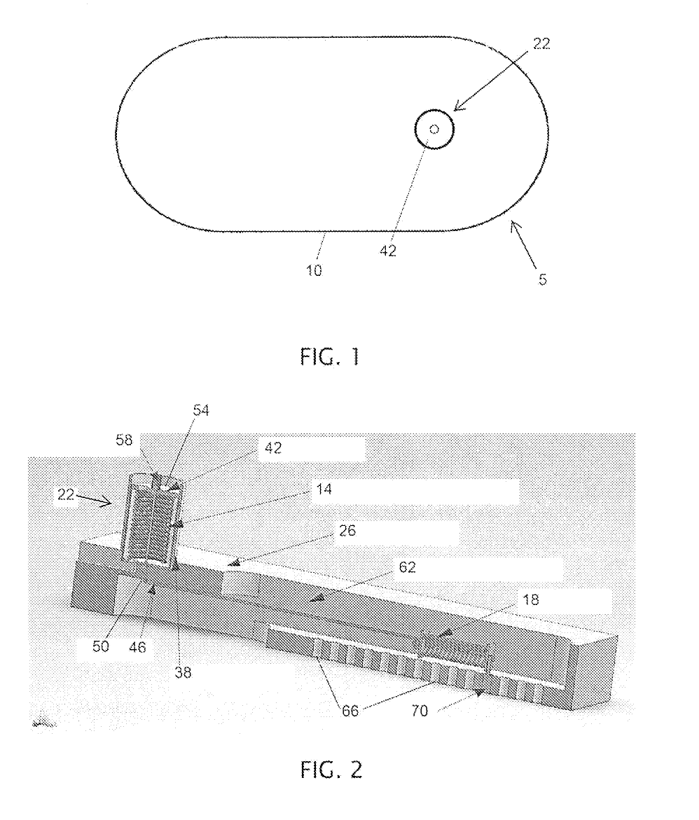

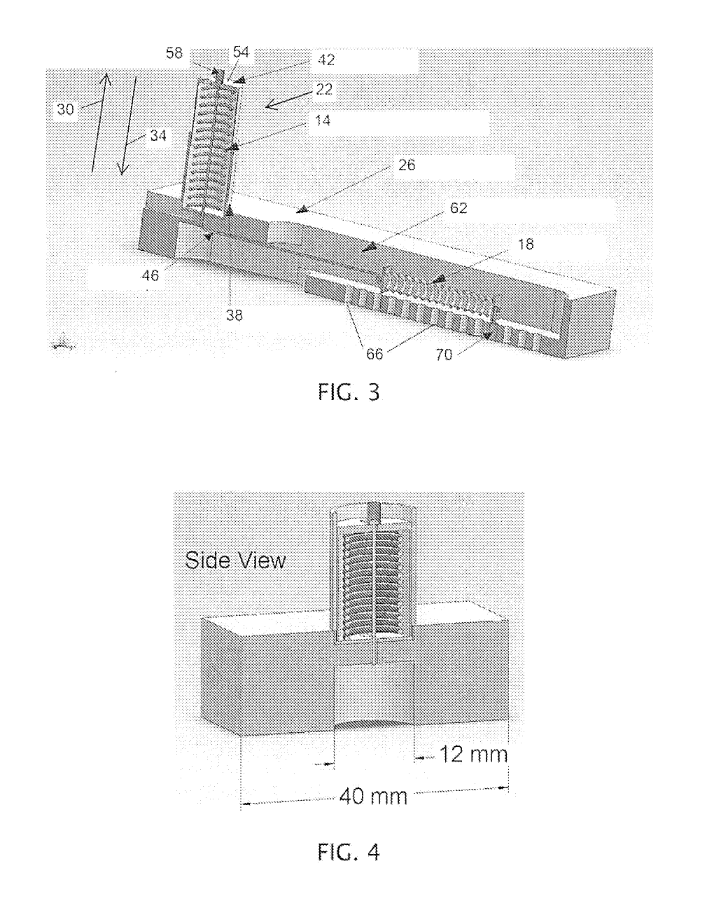

Referring now to the drawings, and more particularly to FIGS. 1-3, shown therein and designated by reference numeral 5 is an embodiment of the present apparatuses (a capsule endoscope having a tool). More particularly, FIGS. 2-3 depict cross-section views of embodiments of the present tool assemblies for use with capsule endoscopes. In the present disclosure, “capsule endoscope” refers to a capsule that is ingestible or otherwise disposable in the digestive tract of an animal (e.g., a human) to investigate or gather data or samples from the digestive tract (e.g., visual inspection, biopsies, sampling of solid or nonsolid tissue or fluid intraluminally and/or extraluminally, etc.). Embodiments of the present actuator assembly can also be used for drug delivery in the gastrointestinal tract from a capsule similar to that of a capsule endoscope. The present actuating assemblies can also be configured to actuate tools to deliver drugs, therapeutic devices, or medical devices into specific sites of the Gastrointestinal tract (e.g., injection or placement of medication, markers, stents, drains, devices, or other diagnostic or therapeutic apparatuses and/or clips or other anchors to keep the device in place). For example, in some embodiments, a hook can be coupled to the tool (e.g., instead of a needle (58) such that a capsule endoscope can anchor itself to tissue (e.g., for a 24-hour period) for monitoring, such as, for example, with one or more additional sensors in a capsule endoscope.

In the embodiment shown, apparatus 5 comprises a capsule 10 configured to be disposed in the digestive tract of an animal (e.g., a human); a shape memory alloy (SMA) element 14 configured to change at least one of its dimensions (e.g., length, as is shown increased or elongated in FIG. 3 relative to FIG. 2) if the temperature of the SMA element is changed (e.g., increased); a biasing element (e.g., spring) 18; and a tool 22 that is coupled to SMA element 14 and spring 18 such that an electrical current can be applied to SMA element 14 to shift tool 22 from a first configuration (FIG. 2) to a second configuration (FIG. 3). The shape or overall size of (e.g., extension and force generation) of the SMA element is generally related to the temperature of the material of the SMA element. Because the SMA element can be activated thermally, numerous ways of activation can be utilized. For example, electric current can be applied to the SMA element to raise the temperature of the element with joule heating, and cooling may occur by natural convection or conduction of thermal energy out of the SMA element). Current can be delivered to the SMA element (material of the SMA element) from a battery power supply using wires, by external induction (an induction source external to the capsule and/or external to the body of a patient in which the capsule is disposed). In the embodiment shown, SMA element 14 is configured to be activated by a low-voltage (3V) direct-current (DC) power supply, which is electrically connected to the SMA material (e.g., by conductive wires). In the embodiment shown, apparatus 5 comprises a tool housing 26 configured to be coupled to capsule 10. In the embodiment shown, SMA element 14 and spring 18 are directly coupled to (and indirectly coupled to capsule 5 via) housing 26. In other embodiments, housing 26 may be unitary with capsule 5. Some examples of shape memory materials that may be used in SMA elements of the present embodiments include: Au—Zd, Cu—Zn, Ni—Ti, Cu—Zn—Al, Ti—Nb, Au—Cu—Zn, Cu—Zn—Sn, Cu—Zn—Si, Cu—Al—Ni, Ag—Cd, Cu—Sn, Cu—Zn—Ga, Ni—Al, Fe—Pt, U—Nb, TI—Pd—Ni, Fe—Mn—Si. Examples of suppliers from which SMA materials can be obtained are: Dynalloy, Inc. (California, USA), Motion Dynamics Corporation (Michigan, USA), and New Horizon Group (Chengdu, China).

In the embodiment shown, SMA element 14 is configured as a coil that is configured to elongate in a first direction 30 if a current is applied (e.g., a DC current of sufficient magnitude, such as in excess of a threshold current), and is configured to retract (or at least be capable of retracting) in a second the direction 34 if the current is removed. In other embodiments, the SMA element can have any suitable shape or configuration (e.g., similar to a leaf spring and/or the like). In the embodiment shown, apparatus 5 is configured such that if tool 22 is in the second configuration (FIG. 3) the current can be removed to shift the tool from the second configuration to the first configuration (FIG. 2). Thus, in the embodiment shown, tool 22 is retracted in the first configuration, and is extended in the second configuration. In the embodiment shown, apparatus 5 is configured such that if tool 22 is in the second configuration (FIG. 3), spring 18 applies a force biasing the tool toward the first configuration (FIG. 2). For example, as SMA element 14 extends and the tool moves from the retracted configuration to the extended configuration, spring 18 is elongated (which in most embodiments will increase the tension between the two ends of the spring). In this way, if the tool is in the extended configuration and the current is removed from SMA element 14, the force applied by spring 18 applies a force tending to return the tool to the retracted configuration.

In the embodiment shown, tool 22, comprises a base 38 and a piston 42, and piston 42 is extended relative to base 38 in the second (extended) configuration (FIG. 3) of the tool. In the embodiment shown, base 38 and piston 42 each have a cylindrical configuration, with the cylindrical portion of base 38 having an inner diameter that is larger than the outer diameter of the cylindrical portion of piston 42, permitting piston 42 to slide relative to base 38. In other embodiments, piston 42 may be larger than base 38 (base 38 may be internal to piston 42), or may have any other suitable configuration that permits the tool to function. In the embodiments shown, SMA element 14 is configured such that application of the current to the SMA element will cause the at least one dimension (e.g., length as shown) of the SMA element to increase and apply a force to piston 42 toward the second (extended) configuration of FIG. 3, and if piston 42 is in the second configuration spring 18 biases the piston toward the first (retracted) configuration. As shown, base 38 is coupled to housing 26. In other embodiments, base 38 may be unitary with housing 26. In the embodiment shown, at least a portion of SMA element 14 is disposed within piston 42 (and/or within base 38). In other embodiments, piston 42 may include a plurality of cylindrical sections with sequentially smaller transverse dimensions (e.g., diameters) that are telescopically (slidingly) coupled together to increase the stroke length of the piston (e.g., similar to an antenna).

In the embodiment shown, apparatus 5 further comprises a cord 46 coupled to and between piston 42 and spring 18. In the embodiment shown, a longitudinal axis of spring 18 is not co-linear with a longitudinal axis of piston 42. Stated another way, spring 18 is not aligned with piston 42. Cord 46 can comprise any suitable line or flexible material permitting apparatus 5 to function as described in this disclosure. For example, in the embodiment shown, cord 46 comprises a suture that can pass through housing 26 at an angle, as shown, and withstand rubbing against housing 26 during extension and retraction of piston 42 relative to base 38. In the embodiment shown, base 38 comprises a an opening or guide hole 50 aligned with a longitudinal axis of piston 42, and cord 46 passes through guide hole 50 and is coupled to the center of a distal end 54 of piston 42. Alignment of guide hole 50 with the longitudinal axis of piston 42, and coupling cord 46 at the center of (distal end of) piston 42 adds stability to piston 42 relative to base 38 by applying the retracting force of spring 18 along the central axis of piston 42 to minimize (e.g., eliminate) lateral forces applied to piston 42 that might otherwise increase the likelihood of buckling or misalignment of piston 42 relative to base 38. In the embodiment shown, base 38 is coupled to piston 42 such that: relative longitudinal motion between piston 42 and base 38 is permitted (e.g., piston 42 can be extended relative to base 38); and relative lateral motion between piston 42 and base 38 is substantially prevented (e.g., piston 42 is substantially constrained to being longitudinally extended and retracted relative to base 38).

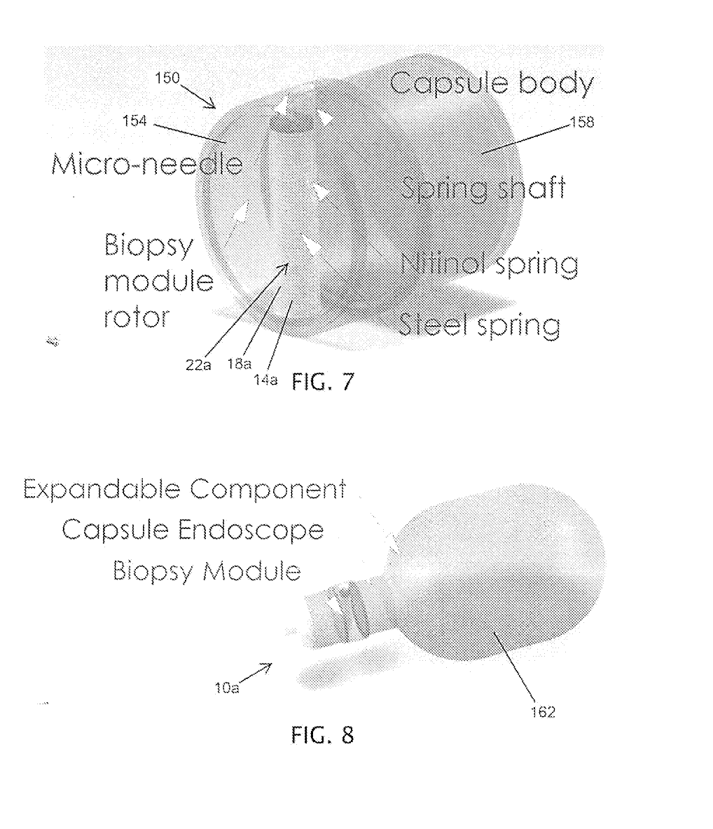

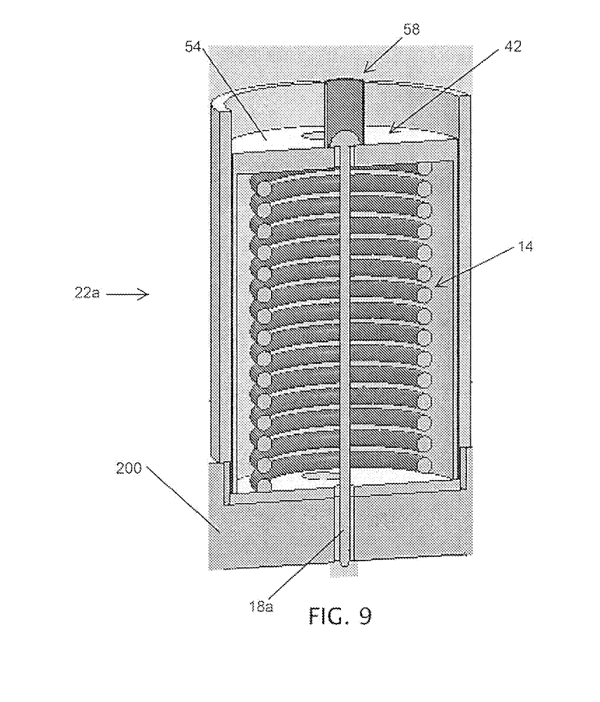

Biasing element 18 can comprise any suitable material or structure to provide a biasing force (e.g., in direction 34) to piston 34 and/or needle 58, such as, for example, to return piston to its first configuration (FIG. 2) as shown. For example, SMA element 14 and biasing element 18 may be coaxial and/or concentric (e.g., with the coil spring that is coaxial and external to SMA element in FIG. 7, and the biasing element inside the SMA element inside the piston, as shown in FIG. 9), or an elastic cord 18a (e.g., of rubber or other elastic material) may be used instead of a spring, as also shown in FIG. 9. In either of these alternative embodiments, cord 46 may be omitted. In such embodiments, for example, a longitudinal or actuation axis of (along with a force is generated by) the biasing element can be parallel to a longitudinal or actuation axis of (along with a force is generated by) the SMA element. By way of another example, biasing element 18 can comprise rubber (e.g., natural or synthetic), resilient polymer or plastic) and/or any other material that enables biasing element 18 to provide a biasing force to piston 42 and/or needle 58, and/or biasing element can have any suitable configuration (e.g., coil spring, cord, and/or the like).

Tool 22 can comprise, for example, a needle 58 coupled to distal end 54 of piston 42. In the embodiment shown, needle 58 is a hollow biopsy needle such that needle 58 can be positioned at a desired position within the digestive tract of an animal and the needle extended into the animal's tissue to extract a biopsy sample of the tissue at the desired location. In other embodiments, needle 58 can be a plain needle, such as may, in some circumstances, be better suited for obtaining fluid samples. In some embodiments, apparatus 5 is configured such that tool 22 (e.g., piston 42) can be actuated to apply a force of at least 20 grams force (go. For example, in some embodiments, apparatus 5 is configured such that piston 42 can be extended (and needle 58 pressed into tissue) with a force of at least 25 gf or more (e.g., 30, 40, 50, 60 gf, or more). In other embodiments, various characteristics of the apparatus may be optimized to reduce the force necessary to actuate a tool, such that a force of less than 20 gf may be sufficient.

In the embodiment shown, housing 26 defines a spring cavity 62 in which spring 18 is disposed. In some embodiments, such as the one shown, housing 26 also includes a plurality of spring-adjustment holes 66 configured to a receive a pin 70 that is coupled to spring 18 (as shown), such that pin 70 can be disposed in one of holes 66 to adjust the resting tension in spring 18 and/or cord 46. For example, holes 66 closer to base 38 will generally result in less tension (up to and including slack in cord 46) than holes 66 relatively farther than base 38.

In the embodiment shown, if a current is applied to (fed through) SMA element 14, SMA element 14 is activated and extends. This extension pushes piston 42 (the inner cylinder), which acts as a low friction carrier for the needle, and causes needle 58 to protrude along the longitudinal axis of the piston (perpendicularly to the surface of the tissue to be sampled). As piston 42 is pushed outwardly relative to base 38, piston 42 pulls cord 46, elongating spring 18. Needle 58 then penetrates the target tissue (e.g., until distal end 54 of piston 42 contacts the surface of the target tissue). The resulting tension in spring 18 (produced by extended SMA element 14) provides a bias or restoring force to pull or retract piston 42 back toward base 38. Cord 46 (suture) helps to provide an axial centering of the forces imparted on piston 42 by SMA element 14 and spring 18 because hole 50 is positioned on a longitudinal axis extending through the center of piston 42 and the cylindrical portion of base 38, thus minimizing friction and buckling. In addition, to minimize buckling, an overlap is provided between base 38 and piston 42, even when piston 42 is in a fully extended position (FIG. 3). Once the current is deactivated or removed from SMA element 14, the elongated (tensioned) spring 18 restores piston 42 to its initial or retracted position, thereby retracting needle 58 with the tissue sample, such that the needled is aligned with the outer edge of the capsule.

In some embodiments, capsule 10 has a length of less than 40 mm and a transverse dimension (e.g., diameter, in the circular embodiment shown) of less than 15 mm. In some embodiments, capsule has a length of less than 32 mm (e.g., 15-20 mm) and a transverse dimension of less than 12 mm (e.g., 6-10 mm). Embodiments of the present apparatuses can also be configured to be used throughout the digestive or gastrointestinal tract (e.g., esophagus, stomach, small intestine, and/or colon). Some embodiments of the present apparatuses can also be configured and/or used for gastrointestinal fluid sampling, small-volume drug delivery, ink injection, and/or marking within the gastrointestinal tract. For example, a drug, ink, and/or marking material (e.g., radioactive or radio-opaque fluid or other material) can be disposed in needle 58 such that at least a first insertion of needle 58 into tissue within the gastrointestinal tract will deliver the drug, ink, and/or marking fluid; and in some embodiments, a second or subsequent insertion of needle 58 into tissue within the gastrointestinal tract will remove a tissue sample for biopsy.

Some embodiments of the present capsules can be configured to adjust the angle of the tool relative to the capsule. For example, as shown in FIG. 7, a capsule endoscope can include a biopsy module comprising a rotor 150 in which tool 22b is disposed and that is rotatably coupled to the capsule. In such embodiments, the biopsy module can also comprise a housing portion 158 including a motor or any other suitable device for rotating rotor 154 to adjust the angle of tool 22a. In the embodiment shown, the biasing element 18a of tool 22a comprises a steel (or other metallic) coil spring disposed around and coaxial to SMA element 14. Some embodiment of the present capsules (e.g., 10a of FIG. 8) can comprise a biopsy module 150 and an expandable element or component 162. Expandable element or component 162 can comprise a balloon or other expandable structure that may, for example, be filled with a sampled fluid within the gastrointestinal tract (e.g., with gastrointestinal fluid drawn into the capsule through a needle (e.g., needle 58) that is in biopsy module and configured to fluidly communicate (e.g., via tubing or the like) with expandable element or component 162. In such embodiments, expandable element or component 162 may be filled with a medication or other fluid (e.g., ink or other marking fluid) that is desired to be delivered to a position in the gastrointestinal tract (e.g., to mark a lesion or the like for identification and/or location during imaging.

The present configurations of SMA element 14, biasing element 18, and/or tool 22 can also be used with other types of endoscopes (e.g., non-capsule endoscopes) for tissue biopsies or fluid sampling (e.g., for obtaining tissue and/or fluid samples from a uterus). For example, in the embodiment of FIG. 7, a tool 22a having a biasing element 18a that comprises an elastic cord is coupled to a body 200 that may be any suitable structure (e.g., a capsule, an endoscope body, a handle for a human hand, and/or the like).

Some embodiments of the present methods comprise: applying an electric current to the SMA element (14) of an embodiment of the present apparatuses (e.g., 5) that is disposed in the digestive tract of an animal (not shown). In some embodiments the tool of the apparatus includes a biopsy needle (e.g., 58), and the method further comprises: actuating the tool to insert the biopsy needle into target tissue of the animal. In some embodiments, the method further comprises: retrieving a tissue sample from the biopsy needle. In some embodiments, the method may include sealing the sample before retrieving the tissue sample (e.g., before removing the capsule from the digestive tract of the animal). For example, embodiments of the apparatus can include a sealing mechanism (e.g., a panel or compartment in the capsule) configure to contain the biopsy tissue after acquisition.

### Maeromodel Assembly and Testing

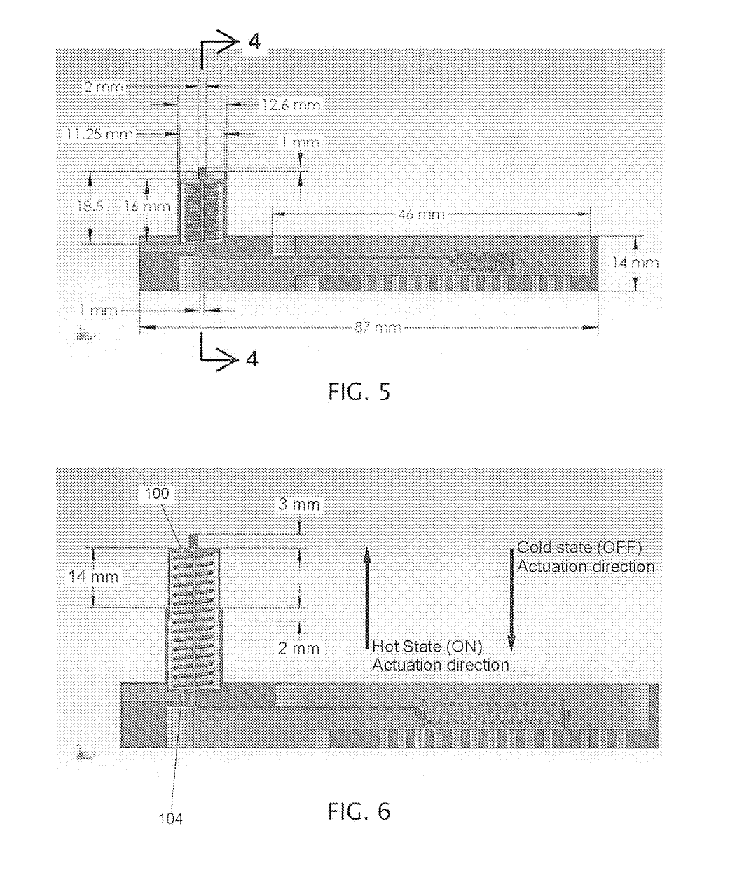

A macromodel of the tool assembly portion of the apparatus shown in FIGS. 2-3 was designed to prove the concept. This tool assembly was manufactured and successfully tested. The system consisted of a housing 26 (comprising acrylic; obtained from Blanson Ltd, Leicester, England, EU), a piston 42 (comprising Delrin; obtained from CONNECTICUT PLASTICS, Wallingford, Conn., USA), a base 38 (also comprising Delrin; also obtained from CONNECTICUT PLASTICS), a needle 58 (comprising surgical steel; available from numerous sources), an SMA element 14 (comprising Nitinol; obtained from Images SI Inc., Staten Island, N.Y., USA), a spring 18 (comprising steel; obtained from Michigan Steel Spring Co., Detroit, Mich., USA), and a cord 46 (comprising poly(p-dioxanone) suture obtained from Ethicon, Inc. in Somerville, N.J., USA). FIGS. 4-6 provide dimensions of the macromodel.

The base (outer cylinder) and piston (inner cylinder) were manufactured from Delrin in the configuration shown, such that the piston could slide inside the base with minimal friction. Delrin was chosen because of its low cost, low friction coefficient, and favorable thermal and mechanical properties. Because Delrin provides favorable thermal isolation, heat generated inside the piston when the SMA element is activated is only minimally transferred to the outer surface of the piston (e.g., such that the temperature of the outer surface of the piston remains in a range that will not cause tissue damage when in use in the digestive tract of a patient). Thus the temperature of the outer surface of the piston is substantially harmless to tissue during a biopsy procedure, and once the SMA element is deactivated, the thermal properties of Delrin do not inhibit cooling of the SMA element. In addition Delrin has a low coefficient of friction, which permits efficient energy transfer (force) from the activated SMA element to the piston without significant friction losses. Delrin is an example of one suitable material, but any materials may be used that enable the apparatus to function as described.

A first power wire (not shown) was inserted through an off-center hole 100 in the distal end of the piston and attached to the top end of the SMA element. A second power wire (not shown) was inserted through an off-center hole 104 in the base and attached to the bottom end of the SMA element. The cord (suture) was then inserted through a central hole in the distal end of the piston, through the middle of the SMA element, through the central holes in the base and the housing, and attached to the steel spring. The base and piston where then press-fit into the housing, as shown. The steel spring was then tensioned to cause the piston to be fully retracted relative to the base, and fixed to a suitable tension hole using a fixation pin. This resulted in the solid height of the SMA element being equal to the inside height of the piston. Stated another way, the bottom of the piston was coincident with the inner face of the base. A surgical needle was then attached to the distal end of the piston. Finally, a power source was connected to the SMA element through an electrical switch for controlling the activation. Multiple currents to the SMA element and tensional forces of the spring were tested to investigate various ranges of currents and tension capable of providing forces sufficient to perform a biopsy. Flexinol® (available from Dynalloy) is one example of a Nitinol material suitable for SMA element 14. The Nitinol wire used for the SMA element of the macromodel had a diameter of 0.008 inches. A Nitinol wire for an embodiment small enough to fit in a capsule endoscope can have a diameter of between 0.005 and 0.006 inches.

Additionally, a separate micro-needle (not attached to the macromodel) was perpendicularly placed with its lower portion onto (in contact with) a stomach model made of silicon (3 mm thick layer) and a minimal force for the micro-needle to penetrate the stomach model was measured by placing different weights on the upper portion of the micro-needle. The results of penetration force were shown to be in a range of 30-35 gf. The macromodel was then tested for the maximum effective force it could exert in a direction perpendicular to the tissue (along the longitudinal axis of the piston). The macromodel could deliver an average maximum force of 540 gf, which must equal or exceed the sum of the biasing (restoring) force from spring 18, and the effective penetration force needed to puncture the tissue. The effective penetrating force ranged between 35-80 gf depending on the activating SMA current. For the present prototype, the targeted penetration force was 35 gf. The maximum current for the SMA element of the prototype produced 80 gf. In the tested macromodel, a current of 900 milliamps (mA) was used. In smaller embodiments (e.g., those small enough to fit in a capsule endoscope), a current of 300-400 mA may be applied to the SMA element. For example, a power source in the capsule can be configured to apply one or more 300-400 mA pulses to the SMA element. The maximum effective fractional stroke (the maximum stroke of the piston from a fully retracted configuration to a fully extended configuration) was 14 mm. In addition, a silicon stomach model having a wall with a 3 mm thickness (three 1 mm layers) was used to verify the biopsy. The activating time for the SMA element to fully extend the needle was 2-3 seconds, and the retracting time for the SMA element to relax and the spring to retract the needle was in the range of 3-4 minutes. The needle successfully punctured the silicon stomach. Further miniaturization of this actuator will allow producing effective force above 50 gf.

### Micromodel Testing

A micro version of the above-described macromodel was also built and tested. Assembly and components were similar to those of the macromodel described above, but with smaller overall dimensions. More particularly, the overall length of tool 22 with piston 42 retracted relative to base 38 was 10 mm; the extended or elongated length of tool 22, with piston 42 extended relative to base 38, was 14 mm; such that the available stroke or deployment length was 4 mm. During testing, a current pulse of 340 mA resulted in an effective force of 40-60 gf with an activated or deployment time of 1 second or less, a retraction time of 2 seconds or less, and a maximum surface temperature of 37° C. or less.

The various illustrative embodiments of devices, systems, and methods described herein are not intended to be limited to the particular forms disclosed. Rather, they include all modifications and alternatives falling within the scope of the claims. For example, the present apparatuses and actuating assemblies can include or be configured to actuate biopsy micro-needles, microspikes (configured to entrap tissue), scoop-like devices, cutting tools and the like. Further, the present actuating assemblies can be configured to actuate tools to deliver drugs, therapeutic devices, or medical devices into specific sites of the Gastrointestinal tract (e.g., injection or placement of medication, markers, stents, drains, devices, or other diagnostic or therapeutic apparatuses and/or clips or other anchors to keep the device in place).

The claims are not intended to include, and should not be interpreted to include, means-plus- or step-plus-function limitations, unless such a limitation is explicitly recited in a given claim using the phrase(s) “means for” or “step for,” respectively.

1-43. (canceled)

44. An apparatus comprising:
a capsule configured to be disposed in the digestive tract of an animal;
a shape memory alloy (SMA) element coupled to said capsule, wherein said SMA element is configured to change at least one of its dimensions if a current is applied to said SMA element;
a biasing element coupled to said capsule; and
a tool coupled to said SMA element and said biasing element such that a current can be applied to said SMA element to shift said tool from a first configuration to a second configuration, and if said tool is in the second configuration the current can be removed to shift said tool from the second configuration to the first configuration.

45. The apparatus of claim 44, wherein a longitudinal axis of said biasing element is parallel to a longitudinal axis of said SMA element.

46. The apparatus of claim 44, wherein said SMA element is configured to expand if a current is applied to said SMA element.

47. The apparatus of claim 46, wherein said capsule further comprises a power source configured to be coupled to said SMA element to apply a current to said SMA element.

48. The apparatus of claim 44, wherein said tool is retracted in the first configuration, and said tool is extended in the second configuration.

49. The apparatus of claim 44, wherein said apparatus is configured such that if said tool is in the second configuration, said biasing element applies a force biasing said tool toward the first configuration.

50. The apparatus of claim 44, wherein said tool comprises a sensor.

51. The apparatus of claim 44, wherein said tool is configured to contact tissue to deliver a therapeutic agent or retrieve a tissue sample.

52. The apparatus of claim 51, wherein said tool comprises a needle.

53. The apparatus of claim 44, wherein said tool comprises a biopsy needle.

54. A method for obtaining a biopsy sample from or administering a therapeutic agent to the digestive tract of a subject, said method comprising:
applying an electric current to said SMA element of an apparatus of claim 44 that is disposed in the digestive tract of a subject, wherein a biopsy needle is coupled to said tool of the apparatus of claim 44;
actuating said tool to insert said biopsy needle into a target area of the subject; and
obtaining a biopsy sample using said biopsy needle.

55. The method of claim 54, wherein said biopsy sample comprises a tissue or a fluid sample.

56. A biopsy apparatus comprising:
a body;
a shape memory alloy (SMA) element coupled to said body, wherein said SMA element is configured to change at least one of its dimensions if a current is applied to said SMA element;
a tool configured to be extended to retrieve a tissue or fluid sample, said tool coupled to said SMA element such that a current can be applied to said SMA element to shift said tool from a first configuration to a second configuration, and if said tool is in the second configuration the current can be removed to shift said tool from the second configuration to the first configuration.

57. The biopsy apparatus of claim 56 further comprising a power source operatively connected to said SMA element and configured to apply a current to said SMA element.

58. The biopsy apparatus of claim 56, wherein said tool comprises a base and a piston, and wherein said piston is extended relative to said base in the second configuration.

59. The biopsy apparatus of claim 58, wherein said SMA element is configured such that application of the current to said SMA element causes at least one dimension of said SMA element to increase and apply a force to said piston toward the second configuration.

60. The biopsy apparatus of claim 56 further comprising a biasing element coupled to said tool and to said body, wherein said biasing element is configured to apply a force urging said tool toward the first configuration.
